(19) United States
(12) Patent Application Publication
(10) Pub. No.: US 2015/0362501 A1
(43) Pub. Date: Dec. 17, 2015

(54) BIOSENSOR AND PROCESS FOR PRODUCING SAME

(71) Applicant: TANAKA KIKINZOKU KOGYO K.K., Tokyo (JP)

(72) Inventors: Masaaki KURITA, Kanagawa (JP); Takashi NISHIMORI, Kanagawa (JP)

(21) Appl. No.: 14/761,456

(22) PCT Filed: Jan. 16, 2014
(86) PCT No.: PCT/JP2014/050722

§ 371 Date: Jul. 16, 2015

(30) Foreign Application Priority Data
Jan. 17, 2013 (JP) 2013-006561

(51) Int. Cl. G01N 33/66; G03F 7/40

(52) U.S. Cl. CPC G01N 33/66; G03F 7/40

(57) ABSTRACT

The present invention provides a biosensor capable of measuring various blood components, in particular, the concentration of blood glucose with high accuracy even when a hematocrit level varies. The above-described object was achieved by a biosensor, which is a biosensor 10 that oxidizes a blood component with an oxidoreductase, detects an oxidation-reduction current generated by the reaction product with an electrode 104 and measures the blood component, and is characterized in that the electrode 104 is an interdigitated array electrode in which a working electrode 1042 and a counter electrode 1044 composed of a noble metal are alternately arranged, the total area of the interdigitated array electrode is from 1.8 to 4 mm2, an inter-electrode distance is less than 50 μm, an electrode width of the working electrode is from 5 to 50 μm, and an electrode width of the counter electrode is from 5 to 100 μm.

### TECHNICAL FIELD

The present invention relates to a biosensor and a method for producing the same, and particularly relates to a biosensor capable of measuring a blood component such as glucose with high accuracy.

### BACKGROUND ART

A biosensor is a sensor which determines the content of a substrate in a sample by utilizing a molecular recognition ability of a biological material such as a microorganism, an enzyme, an antibody, a DNA or an RNA. Among various biosensors, a sensor utilizing an enzyme has been in practical use, and for example, glucose, lactic acid, cholesterol, amino acids, and the like in a substrate can be measured.

As a biosensor for measuring blood glucose levels, which is one of the representative biosensors, there has been a biosensor which mainly utilizes an electrochemical reaction, uses, for example, a reagent such as potassium ferricyanide as a mediator, causes glucose in blood and an enzyme such as glucose oxidase carried in the sensor to react with each other, and measures the obtained current value, thereby determining blood glucose levels (see, for example Patent Document 1).

On the other hand, as an index for the viscosity of blood, there has been known a hematocrit level. The hematocrit level is a ratio (%) of the volume of red blood cells in blood, and is generally from 40 to 50% in healthy adults. On the other hand, the hematocrit level decreases in anemia patients, and there is also a case that anemia patients are put into a state where the hematocrit level is lower than 15%. Such a variation in hematocrit level is known to adversely affect the determination of the concentration of a blood component, particularly glucose using a biosensor. However, any conventional techniques cannot cope with such a variation in hematocrit level and have a problem with measurement accuracy of the concentration of blood glucose.

### CITATION LIST

### Patent Documents

Patent Document 1: JP-T-2005-512027

### SUMMARY OF THE INVENTION

### Problems to be Solved by the Invention

In view of this, an object of the present invention is to provide a biosensor capable of measuring various blood components, in particular, the concentration of blood glucose with high accuracy even when a hematocrit level varies, and a method for producing the same.

### Means for Solving the Problems

As a result of intensive studies, the present inventors found that conventional problems as described above can be solved by using an interdigitated array electrode having a specific total area, a specific inter-electrode distance and a specific electrode width, or further having a specific number of electrodes as an electrode in a biosensor utilizing an electrochemical reaction, and thus, could complete the present invention.

That is to say, the present invention is as follows.
1. A biosensor which oxidizes a blood component with an oxidoreductase, detects an oxidation current generated by the reaction product with an electrode and measures the blood component, wherein the electrode is an interdigitated array electrode in which a working electrode and a counter electrode composed of a noble metal are alternately arranged, the total area of the interdigitated array electrode is from 1.8 to 4 mm², an inter-electrode distance is less than 50 μm, an electrode width of the working electrode is from 5 to 50 μm and an electrode width of the counter electrode is from 5 to 100 μm.
2. The biosensor described in 1 above, wherein the sum of the number of the working electrodes and the counter electrodes of the interdigitated array electrode is from 30 to 300.
3. The biosensor described in 1 or 2 above, wherein the interdigitated array electrode is (1) formed by forming a noble metal film on an electrically insulating substrate, printing a resist in the form of an interdigitated array thereon by a screen printing method, performing etching, followed by removing the resist, or (2) formed by forming a noble metal film on an electrically insulating substrate, applying or adhering a resist thereon, performing light exposure through a photomask, etching the resist and the noble metal film in a portion other than a portion where the interdigitated array electrode is formed, followed by removing the resist in the portion where the interdigitated array electrode is formed, or (3) formed by superimposing a template from which a pattern of the interdigitated array electrode to be produced has been removed on an electrically insulating substrate, forming a noble metal film on the electrically insulating substrate through the template, followed by removing the template, or (4) formed by printing a resist in a portion where the interdigitated array electrode is not formed on an electrically insulating substrate by a screen printing method, forming a noble metal film on the electrically insulating substrate and the resist and removing the resist and the noble metal film formed on the resist.
4. The biosensor described in any one of 1 to 3 above, wherein the blood component is glucose.
5. A method for producing a biosensor, comprising a step of forming an interdigitated array electrode, in which a working electrode and a counter electrode composed of a noble metal are alternately arranged, on an electrically insulating substrate, wherein the total area of the interdigitated array electrode is from 1.8 to 4 mm², an inter-electrode distance is less than 50 μm, an electrode width of the working electrode is from 5 to 50 μm, an electrode width of the counter electrode is from 5 to 100 μm and the number of the electrodes is from 30 to 300, the step is (1) a step of forming an interdigitated array electrode by forming a noble metal film on an electrically insulating substrate, printing a resist in the form of an interdigitated array thereon by a screen printing method, performing etching, followed by removing the resist, or (2) a step of forming an interdigitated array electrode by forming a noble metal film on an electrically insulating substrate, applying or adhering a resist thereon, performing light exposure through a photomask, etching the resist and the noble metal film in a portion other than a portion where the interdigitated array electrode is formed, followed by removing the resist in the portion where the interdigitated array electrode is formed, or (3) a step of forming an interdigitated array electrode by superimposing a template from which a pattern of the interdigitated array electrode to be produced has been removed on an electrically insulating substrate, forming a noble metal film on the electrically insulating substrate through the template, followed by removing the template.

### Effect of the Invention

According to the present invention, since an interdigitated array electrode having a specific total area, a specific inter-electrode distance and a specific electrode width, or further having a specific number of electrodes is used as an electrode in a biosensor utilizing an electrochemical reaction, an electric double layer which is less affected by hematocrit is formed, and also a current value generated by a redox reaction sufficient for measurement is obtained in a short time, and a blood component such as glucose can be measured.

Accordingly, a biosensor capable of measuring various blood components with high accuracy even when a hematocrit level in blood varies, and a method for producing the same can be provided. For example, the contents of glucose, lactic acid, cholesterol, and the like contained in blood can be measured with high accuracy.

### BRIEF DESCRIPTION OF THE DRAWINGS

FIG. 1 is an exploded perspective view showing one example of a biosensor of the present invention.

FIG. 2 is a plan view for illustrating an interdigitated array electrode to be used in the present invention.

FIGS. 3(a) to 3(e) are views showing a step of producing an interdigitated array electrode by a method using a printing mask formed by screen printing.

FIGS. 4(a) to 4(g) are views showing a step of producing an interdigitated array electrode by a method using a mask formed by photolithography.

FIGS. 5(a) to 5(e) are views showing a step of producing an interdigitated array electrode by a method using a metal mask.

FIGS. 6(a) to 6(d) are views showing measurement results of current values in Example 1.

FIG. 7 is a view showing CV values calculated at each sampling time in Example 1.

FIGS. 8(a) to 8(d) are views showing results of performing chronoamperometry in Example 1.

FIGS. 9(a) to 9(c) are views showing changes in current values when using Ht42 as a reference in FIG. 8.

FIG. 10 is a view showing results of performing chronoamperometry in Example 2.

FIGS. 11(a) to 11(c) are views showing the effect of Ht calculated from FIG. 10.

FIGS. 12(a) to 12(d) are views showing a step of producing an interdigitated array electrode by a lift-off method.

### EMBODIMENTS FOR CARRYING OUT THE INVENTION

Hereinafter, the present invention will be described in more detail.

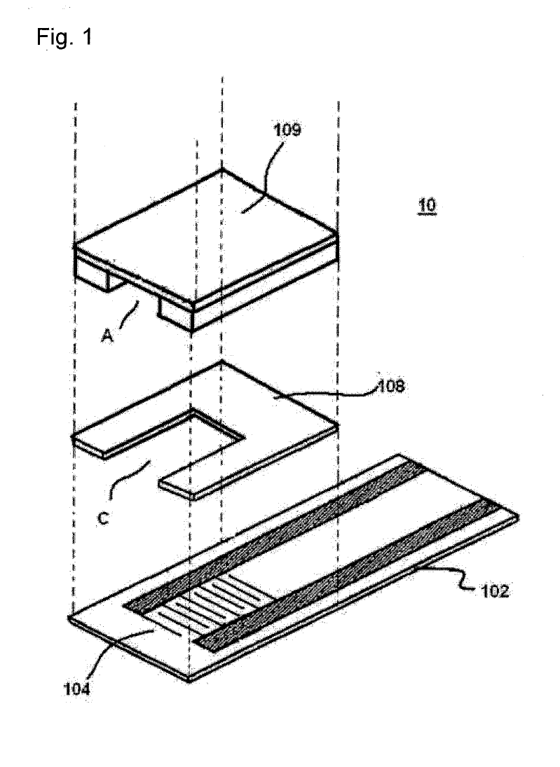

FIG. 1 is an exploded perspective view showing one example of a biosensor of the present invention. In FIG. 1, a biosensor 10 oxidizes a blood component with an oxidoreductase, detects an oxidation current generated by the reaction product with an electrode and measures the blood component. Specifically, an interdigitated array electrode 104 is formed on an electrically insulating substrate 102, a reagent layer (not shown) is provided on the interdigitated array electrode 104, and a spacer 108 is further provided thereon by, for example, printing, whereby the total area of the interdigitated array electrode 104 is defined. Further, on the spacer 108, a cover film 109 is provided. The spacer 108 is provided with a notch in a portion corresponding to the interdigitated array electrode 104 and the reagent layer to form a cavity C.

Examples of materials for forming the electrically insulating substrate 102, the spacer 108 and the cover film 109 include polyester, polyolefin, polyamide, polyesteramide, polyether, polyimide, polyamide-imide, polystyrene, polycarbonate, poly-ρ-phenylene sulfide, polyether ester, polyvinyl chloride and poly(meth)acrylic acid ester. In particular, a film composed of polyester, for example, polyethylene terephthalate, polyethylene-2,6-naphthalate, polybutylene terephthalate, or the like is preferred.

The reagent layer provided on the interdigitated array electrode 104 contains an oxidoreductase, a redox mediator, a hydrophilic polymer, and the like. The oxidoreductase and the redox mediator may be appropriately selected according to the type of the blood component to be measured, however, examples of the oxidoreductase include glucose oxidase, lactate oxidase, cholesterol oxidase, cholesterol esterase, uricase, ascorbate oxidase, bilirubin oxidase, glucose dehydrogenase, lactate dehydrogenase and lactate dehydrogenase. Examples of the redox mediator include potassium ferricyanide, p-benzoquinone or a derivative thereof, phenazine methosulfate, methylene blue and ferrocene or a derivative thereof. Examples of the hydrophilic polymer include carboxymethyl cellulose.

When a blood component is measured, blood in an amount of less than 1 μL, for example, 0.1 to 0.25 μL is introduced into a hole A of the cover film 109, and guided to a position where the interdigitated array electrode 104 and the reagent layer are placed. Then, a current value generated by the reaction between the blood and the reagent on the interdigitated array electrode 104 is read by an external measurement device through a lead (not shown).

The configuration of the biosensor described above is known, however, in a conventional biosensor, when a hematocrit level varies, the determination of a blood component, particularly glucose is adversely affected. Therefore, in order to solve this problem, the present invention is characterized by using an interdigitated array electrode having a specific total area, a specific inter-electrode distance and a specific electrode width, or further having a specific number of electrodes.

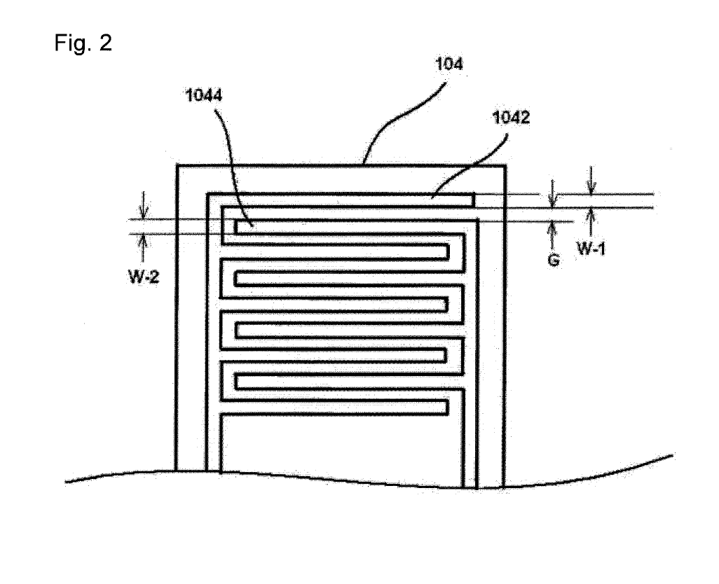

FIG. 2 is a plan view for illustrating the interdigitated array electrode to be used in the present invention. In FIG. 2, the interdigitated array electrode 104 has a configuration in which each of a working electrode 1042 and a counter electrode 1044 is formed into a comb shape, and the working electrode 1042 and the counter electrode 1044 are disposed facing each other so that the teeth portions of the comb shapes are alternately interdigitated with each other.

The interdigitated array electrode 104 to be used in the present invention is characterized in that the total area is from 1.8 to 4 mm², an inter-electrode distance G is less than 50 μm, an electrode width W-1 of the working electrode 1042 is from 5 to 50 μm, and an electrode width W-2 of the counter electrode 1044 is from 5 to 100 μm, or is further characterized in that the number of the electrodes is from 60 to 300. The total area as used herein refers to the total area of portions which are not covered with the spacer 108 of the teeth portions of the comb shapes of the working electrode 1042 and the counter electrode 1044. Further, the number of the electrodes refers to the sum of the number of the teeth of the comb shapes of the working electrode 1042 and the counter electrode 1044.

If the total area is less than 1.8 mm², a signal becomes weak, while if it exceeds 4 mm², not only the effect of hematocrit cannot be sufficiently suppressed, but also the amount of blood to be collected is increased to increase the burden on patients, and therefore, such a total area is not preferred.

If the inter-electrode distance G is 50 μm or more, the effect of hematocrit cannot be sufficiently suppressed, and therefore, such an inter-electrode distance is not preferred.

If the electrode width W-1 of the working electrode 1042 is less than 5 μm, a signal becomes weak, while if it exceeds 50 μm, the effect of hematocrit cannot be sufficiently suppressed, and therefore, such an electrode width is not preferred.

If the electrode width W-2 of the counter electrode 1044 is less than 5 μm, a signal becomes weak, while if it exceeds 100 μm, the effect of hematocrit cannot be sufficiently suppressed, and therefore, such an electrode width is not preferred.

From the viewpoint of enhancing the effect of the present invention, the interdigitated array electrode 104 to be used in the present invention is more preferably configured such that the total area is from 1.8 to 3.0 mm², the inter-electrode distance G is from 5 to 30 μm, the electrode width W-1 of the working electrode 1042 is from 5 to 30 μm, the electrode width W-2 of the counter electrode 1044 is from 5 to 70 μm, and the number of the electrodes is from 150 to 300.

Further, examples of the noble metal constituting the interdigitated array electrode 104 include gold, silver, platinum, palladium, rhodium, iridium, ruthenium and osmium, however, from the viewpoint of enhancing the effect of the present invention, gold is preferred.

The interdigitated array electrode 104 to be used in the present invention can be formed by, for example, the following methods.

(1) Method using printing mask formed by screen printing

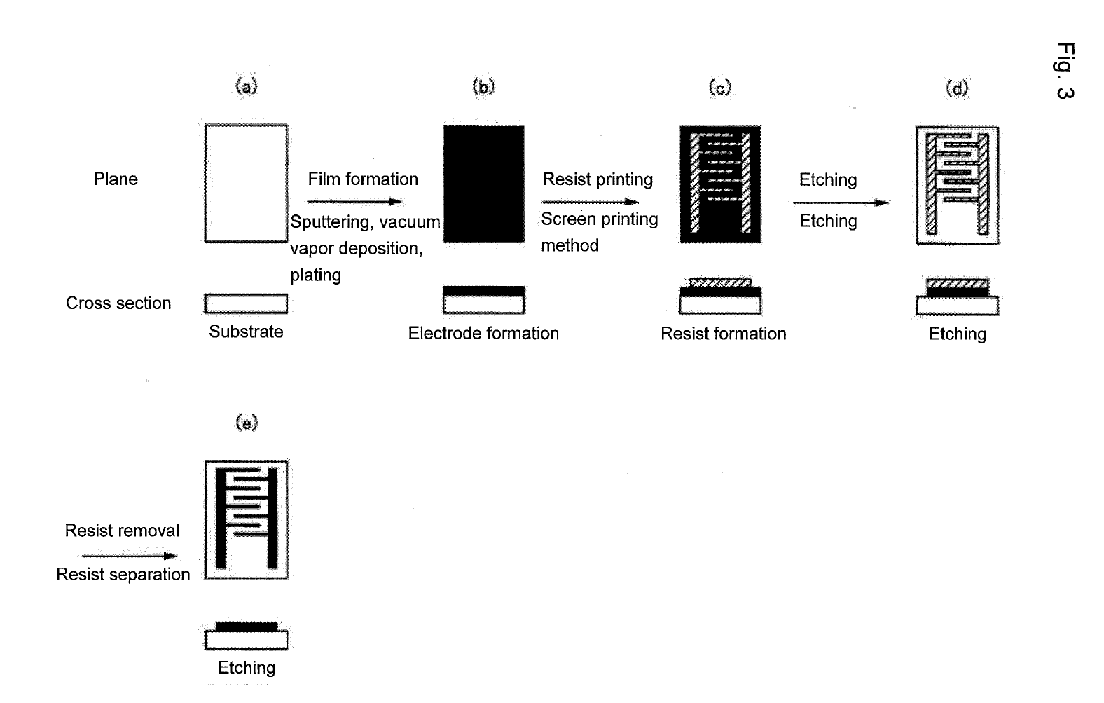

FIG. 3 is a view showing a step of producing the interdigitated array electrode 104 by a method using a printing mask formed by screen printing.

First, an electrically insulating substrate is prepared [FIG. 3(a)], and a noble metal film is formed on the electrically insulating substrate by a means such as sputtering, vacuum vapor deposition or plating of a noble metal constituting the interdigitated array electrode [FIG. 3(b)].

Subsequently, a resist is printed in the form of an interdigitated array on the electrode film by adopting a screen printing method [FIG. 3(c)], and etching is performed [FIG. 3(d)].

Finally, the resist is removed by a stripping solution or the like, whereby the interdigitated array electrode is completed [FIG. 3(e)].

(2) Method using mask formed by photolithography

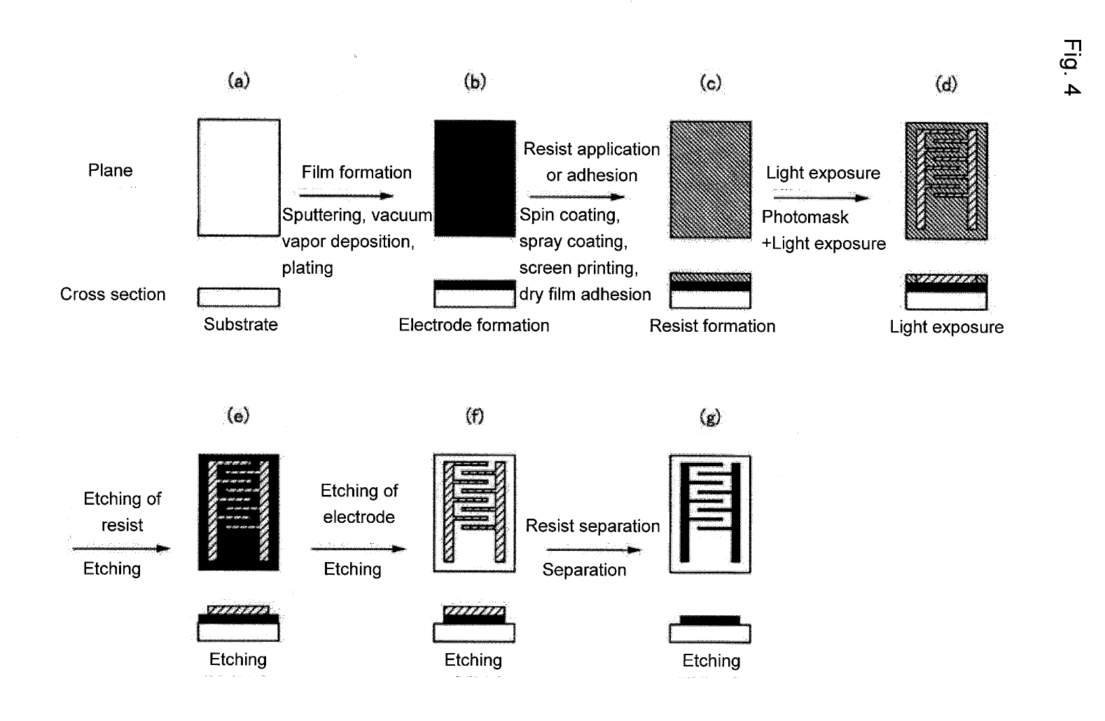

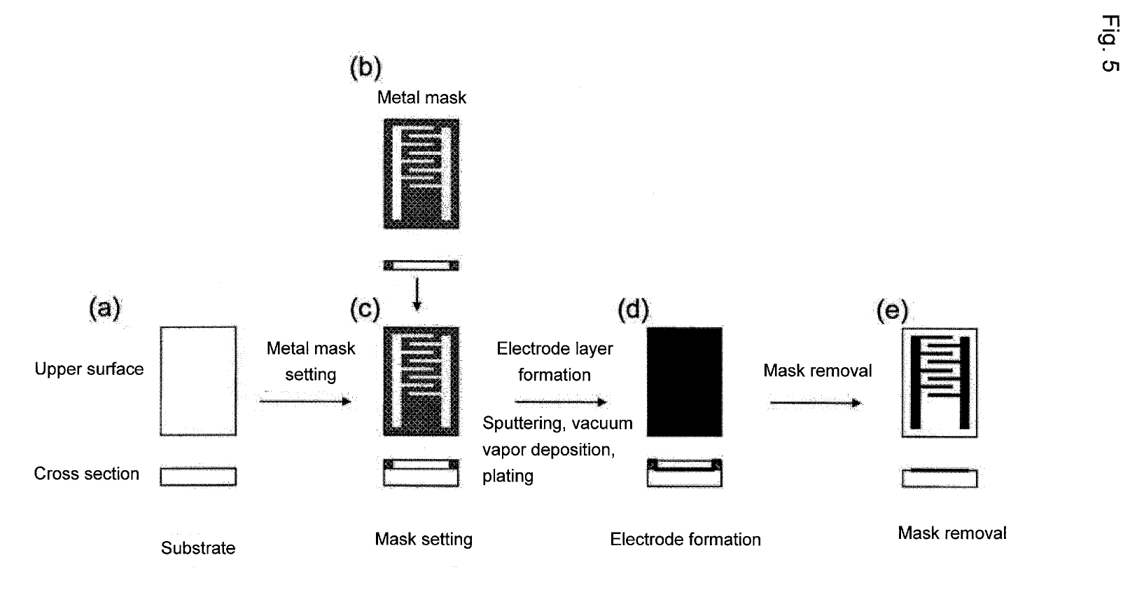

FIG. 4 is a view showing a step of producing the interdigitated array electrode 104 by a method using a mask formed by photolithography

First, an electrically insulating substrate is prepared [FIG. 4(a)], and a noble metal film is formed on the electrically insulating substrate by a means such as sputtering, vacuum vapor deposition or plating of a noble metal constituting the interdigitated array electrode [FIG. 4(b)].

Subsequently, a resist is applied or adhered on the noble metal film by adopting a means such as spin coating, spray coating, screen printing or dry film adhesion [FIG. 4(c)], and light exposure is performed through a photomask [FIG. 4(d)].

Subsequently, the resist and the noble metal film in a portion other than a portion where the interdigitated array electrode is formed are etched [FIGS. 4(e) and 4(f)].

Finally, the resist in the portion where the interdigitated array electrode is formed is removed by a stripping solution or the like, whereby the interdigitated array electrode is completed [FIG. 4(g)].

(3) Method using metal mask

FIG. 5 is a view showing a step of producing the interdigitated array electrode 104 by a method using a metal mask.

First, an electrically insulating substrate is prepared [FIG. 5(a)], and a template from which a pattern of the electrode to be produced has been removed (called “metal mask”) [FIG. 5(b)] is superimposed on the substrate [FIG. 5(c)], and then, the electrode is formed by a treatment with a means such as sputtering, vacuum vapor deposition or plating of a noble metal constituting the electrode [FIG. 5(d)], whereby a noble metal film is formed on the electrically insulating substrate. Subsequently, the metal mask is removed, whereby the electrode is completed [FIG. 5(e)].

(4) Lift-Off Method

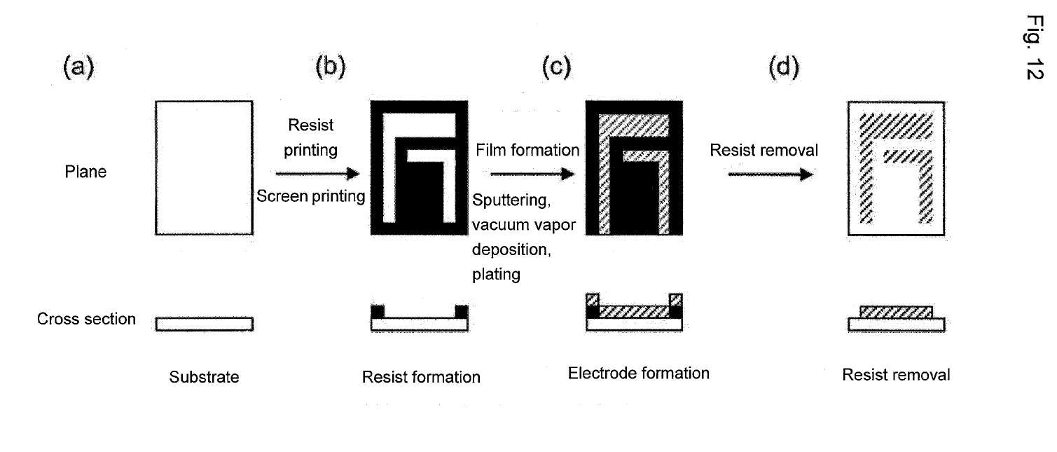

FIG. 12 is a view showing a step of producing the interdigitated array electrode 104 by a lift-off method.

First, an electrically insulating substrate is prepared [FIG. 12(a)], and a resist is printed in the form of a flat plate in a portion where the electrode is not formed by adopting a screen printing method [FIG. 12(b)], followed by drying.

Subsequently, on the substrate having the resist printed thereon, a noble metal film is formed by a means such as sputtering, vacuum vapor deposition or plating of a noble metal constituting the electrode [FIG. 12(c)].

Finally, the resist and the noble metal film formed on the resist are removed by removing the resist with a stripping solution or the like, whereby the electrode is completed [FIG. 12(d)].

In the present invention, from the viewpoint that a desired interdigitated array shape can be formed with high accuracy and less irregularities on the surface including an electrode edge portion, it is preferred to adopt the method using a mask formed by photolithography in the above (2).

### EXAMPLES

Hereinafter, the present invention will be further described with reference to Examples and Comparative Examples, however, the present invention is not limited to the following examples.

### Example 1

Purpose: Evaluation of gold interdigitated array electrode formed by photolithography

1. Measurement of CV value

2. Examination regarding effect of different hematocrit levels (hereinafter referred to as “Ht levels”) on sensor response:

Evaluation of gold interdigitated array electrode using Ht derived from horse blood in homogeneous solution system

Experiment:

Evaluation of gold interdigitated array electrode produced by method using mask formed by photolithography

Three gold interdigitated array electrodes (IDA) with a spacer produced by photolithography were prepared.

(1) 20 μm IDA (width of working electrode/width of counter electrode/inter-electrode distance=20 μm/20 μm/20 μm, sum of number of working electrodes and counter electrodes=72, total area of electrode including working electrodes and counter electrodes=2.2 mm²)

(2) 50 μm IDA (width of working electrode/width of counter electrode/inter-electrode distance=50 μm/50 μm/50 μm, sum of number of working electrodes and counter electrodes=28, total area of electrode including working electrodes and counter electrodes=2.0 mm²)

(3) 80 μm IDA (width of working electrode/width of counter electrode/inter-electrode distance =80 μm/80 μm/80 μm, sum of number of working electrodes and counter electrodes=18, total area of electrode including working electrodes and counter electrodes=2.2 mm²)

Further, one gold interdigitated array electrode (IDA) with a spacer produced by a method using a printing mask formed by screen printing was prepared.

(4) printing mask 50 μm IDA (width of working electrode/width of counter electrode/inter-electrode distance=50 μm/50 μm/50 μm, sum of number of working electrodes and counter electrodes=28, total area of electrode including working electrodes and counter electrodes=2.0 mm²)

On each of these electrodes, a seal (cover film) which forms a capillary with a volume of 0.8 μL (5×2×0.08 mm³) was adhered, thereby forming a capillary, and the following examinations were performed.

1. Measurement of CV value

A solution of potassium ferrocyanide at a final concentration of 10 mM, potassium ferricyanide at a final concentration of 90 mM and potassium phosphate buffer at a final concentration of 100 mM (hereinafter referred to as “P.P.B”) (pH 7.5) was prepared. The thus prepared mixed solution was applied to the capillary on the electrode at 0 V vs. CCP. At 5 seconds after the application to the electrode, a potential of +200 mV was applied, and a current value was measured for 20 seconds (the measurement was performed under the following condition: sampling at 10 Hz (10 points/sec)).

The measurement was performed under the same condition using 10 electrodes and a CV value ((standard deviation/average)×100) was calculated from the obtained current values.

2. Effect of Ht on current value in homogeneous solution system using Ht derived from horse blood

Preserved horse blood (Nippon Biotest Laboratories Inc., Cat. No. 0103-1) was washed 5 times with PBS(−) (1000 g, 10 min). To the washed blood sample, a substrate adjusted with phosphate buffered saline (hereinafter referred to as “PBS(−)”) so that the concentration in the liquid component was 571.4 mg/dL glucose was added, whereby an Ht30 sample was prepared.

The Ht30 sample was centrifuged (1000 g, 4° C., 10 min), and the resulting supernatant was partially removed, whereby Ht56, Ht49, Ht42 and Ht21 samples were prepared. The supernatant obtained by centrifugation was used as an Ht0 sample. To the samples other than Ht0, a glucose solution adjusted with PBS(−) was added, and preparation was performed so that the final concentration in the liquid component in the reaction solution was 400 mg/dL.

An enzyme-mediator mixed solution was prepared so that the final concentrations of the respective components in the reaction solution were as follows: flavin adenine dinucleotide-dependent glucose dehydrogenase (hereinafter referred to as “FADGDH”) at 1 U/μL (calculated from an activity value at 40 mM glucose in a PMS-DCIP system using phenazine methosulfate (PMS) and 2,6-dichlorophenol indophenol (DCIP)), 100 mM potassium ferricyanide and 100 mM P.P.B. (pH 7.5). To 1.5 μL of this mixed solution, 3.5 μL of the substrate-Ht solution containing 400 mg/dL glucose and Ht0, Ht21, Ht42, Ht49 or Ht56 prepared as described above was added, whereby the reaction solution was prepared. The reaction solution was added to the capillary, a potential of +200 mV was applied, and a current value was measured for 20 seconds (before performing the measurement, 0 V vs. CCP was applied for 5 seconds, and the measurement was performed under the following condition: sampling at 10 Hz (10 points/sec)).

### RESULTS

1. Measurement of CV value of gold interdigitated array electrode (hereinafter also referred to as “IDA”) produced by photolithography

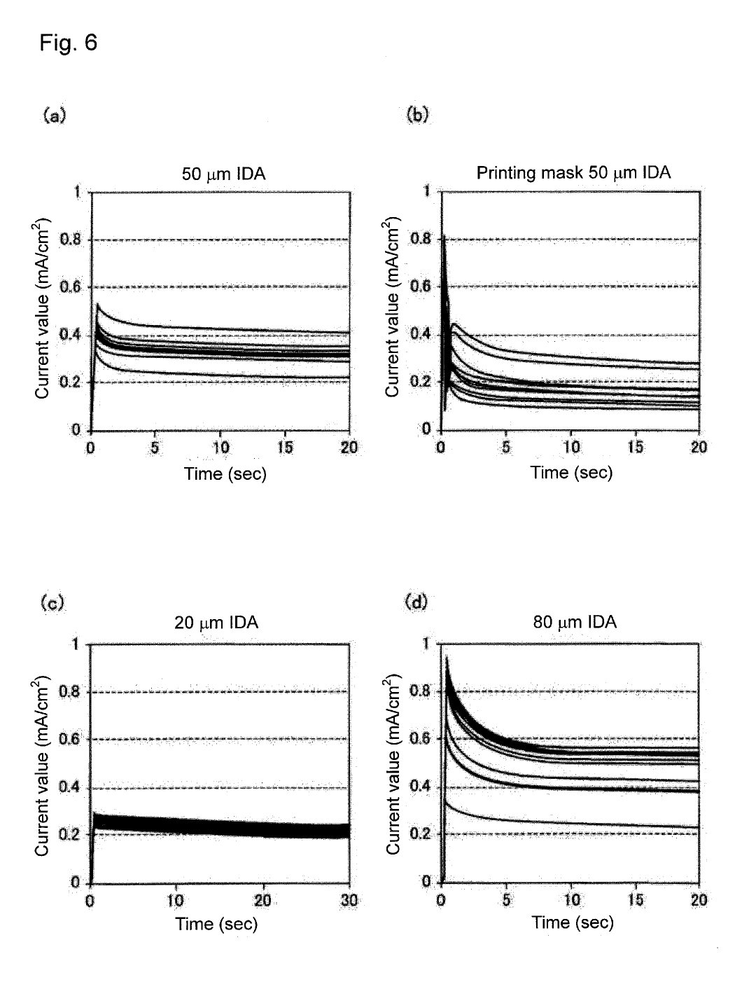

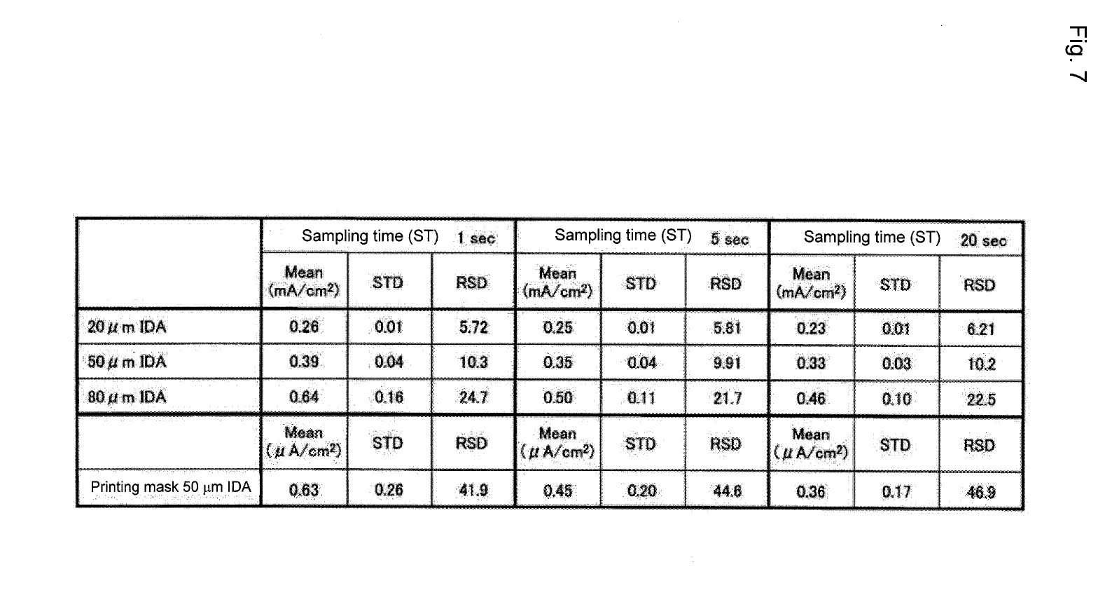

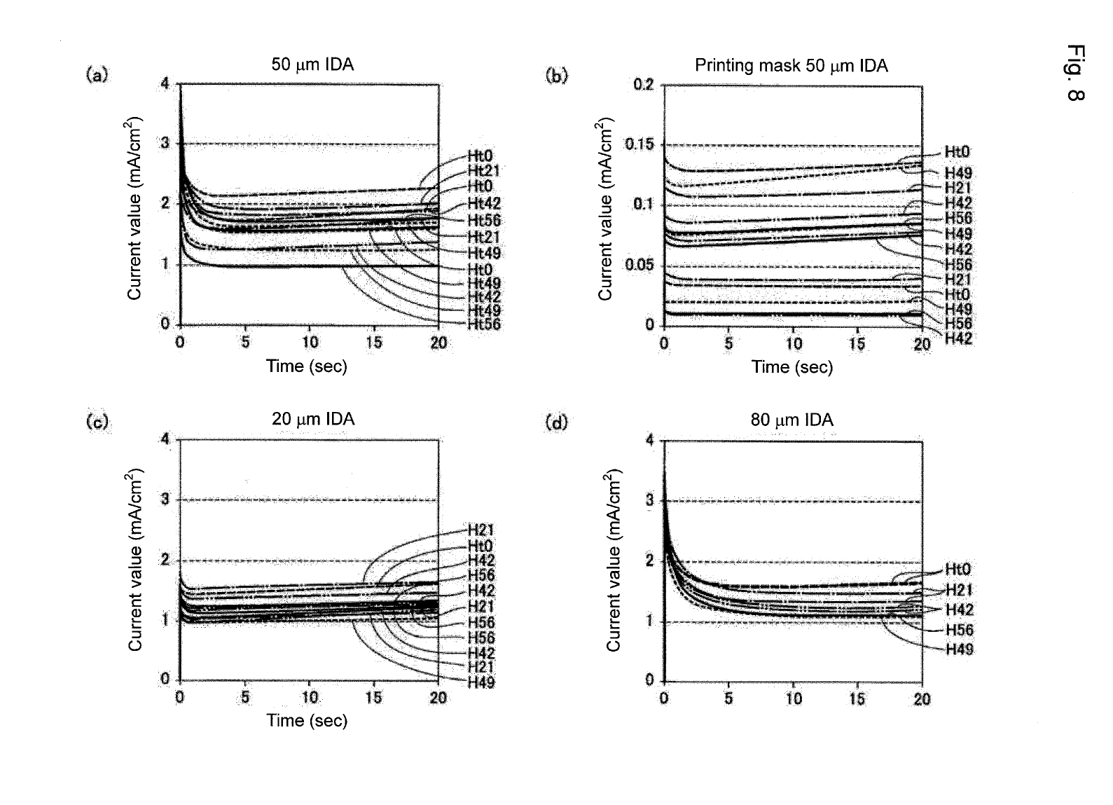

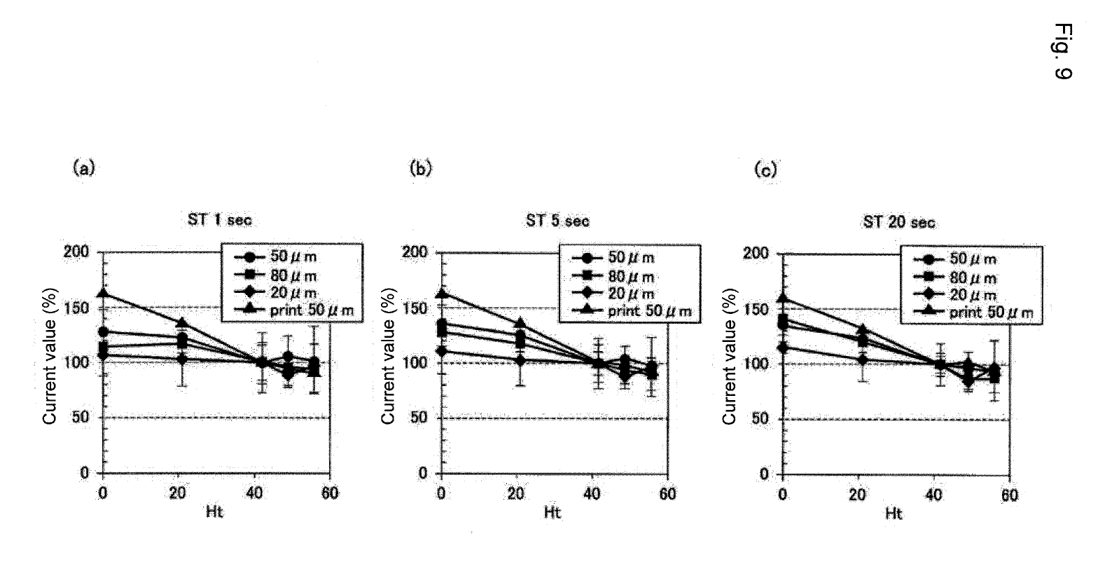

The measurement results of the current values are shown in FIGS. 6(a) to 6(d), and the CV values calculated at each sampling time are shown in FIG. 7.

When comparing the 50 μm IDA and the printing mask 50 μm IDA, the shapes of curves of amperograms are different, and it is found that a plateau region is reached faster in the case of the electrode produced by photolithography (50 μm IDA). In the case of the printing mask 50 μm IDA, many electrodes showed a curve with two peaks.

On the other hand, when comparing the 20 μm IDA, 50 μm IDA and 80 μm IDA, as the electrode width was smaller, the current value reached a plateau region faster, and in the case of 20 μm, the current value became substantially constant after 1 second, however, in the case of 80 μm, it took 5 seconds or more to reach a plateau region. The current value in a plateau region was higher as the electrode width was larger.

With respect to the CV value, in the case of the electrodes produced by photolithography, there was no difference in values calculated at any sampling time, and the 20 μm IDA had a CV value of about 6, which is the lowest, the 50 μm IDA had a CV value of about 10, and the 80 μm IDA had a CV value of around 23. While the 50 μm IDA had a CV value of about 10, the printing mask 50 μm IDA had a CV value of 40 or more, which was considerably high.

The number of the electrodes used for calculating the CV value in this test was 10, and a possibility that the calculated CV value is somewhat higher than the actual CV value is high. Further, when calculation is performed by excluding the results of only one electrode deviated from the other results in the case of the 50 μm IDA, the CV value thereof is similar to that of the 20 μm IDA.

From the above results, it was shown that the current value varies depending on the method for producing the electrode and the reproducibility of the electrode produced by photolithography is high, and also it was revealed that the performance of the electrode produced by photolithography is high.

2. Effect of Ht (hematocrit) on current value in IDA electrode produced by photolithography (homogeneous solution system)

The results of performing chronoamperometry by mixing the enzyme-mediator mixed solution and the Ht0 to Ht56 substrate solution are shown in FIGS. 8(a) to 8(d), and changes in current values when using Ht42 as a reference are shown in FIGS. 9(a) to 9(c).

From the amperograms, it was shown that as the electrode width of the IDA is smaller, a plateau region is reached faster. The current value of the electrode (50 μm IDA) produced by photolithography was approximately 1.5 mA/cm², and the electrodes having a different electrode width also showed a nearly equal current density. On the other hand, the current density measured in the printing mask 50 μm IDA was 1/10 or less of that of the electrode produced by photolithography.

The effect of Ht was the smallest in the case of the 20 μm IDA, and in the case of the 50 μm IDA and the 80 μm IDA, substantially the same effect of Ht was observed. In particular, in the case of the 20 μm IDA, a change in the current value was about ±10% in the range between Ht20 and Ht56, and the effect of Ht was small.

### Example 2

Purpose:

1. Examination of effect of Ht on IDA electrode produced by photolithography (dry chip)

Experiment:

1. Examination of effect of Ht on IDA electrode produced by photolithography

An IDA electrode (width of working electrode/width of counter electrode/inter-electrode distance=30 μm/30 μm, sum of number of working electrodes and counter electrodes=48, total area of electrode=2.2 mm²) with a spacer produced by photolithography was produced, and the following examination was performed.

Preserved horse blood (Nippon Biotest Laboratories Inc., Cat. No. 0103-1) was washed 5 times with PBS(−) by PBS(−) (1500 g, 10 min). To the washed blood sample, a substrate adjusted with PBS(−) so that the final concentration in the liquid component was 400 mg/dL glucose was added, whereby an Ht40 sample was prepared. The Ht40 sample was centrifuged (1000 g, 4° C., 10 min), and the resulting supernatant was added or partially removed, whereby Ht20, Ht30, Ht40, Ht50 and Ht60 samples were prepared.

An enzyme-mediator solution was prepared so as to contain FADGDH at 2 U/μL (calculated from an activity value at 40 mM glucose in a PMS-DCIP system), 200 mM potassium ferricyanide, 50 mM sucrose, 0.3% Lucentite and 100 mM P.P.B. (pH 7.5) at the time of condensation, and 1 μL of the thus prepared solution was applied on the electrode and dried at 37° C. for 10 min and at 50° C. for 5 min. To this dried chip (dry chip), a seal (cover film) which forms a 0.8-μL capillary was adhered, whereby a dry chip for measurement was produced.

To the produced dry chip, the substrate-Ht solution containing 400 mg/dL glucose and Ht20, Ht30, Ht40, Ht50 or. Ht60 prepared as described above was added. At 5 seconds after the addition of the substrate, a potential of +200 mV was applied, and a current value was measured for 30 seconds (0 V vs. CCP was applied during a waiting time (WT), sampling: 10 Hz (10 points/sec)).

### RESULTS

1. Examination of effect of Ht on IDA electrode produced by photolithography (dry chip)

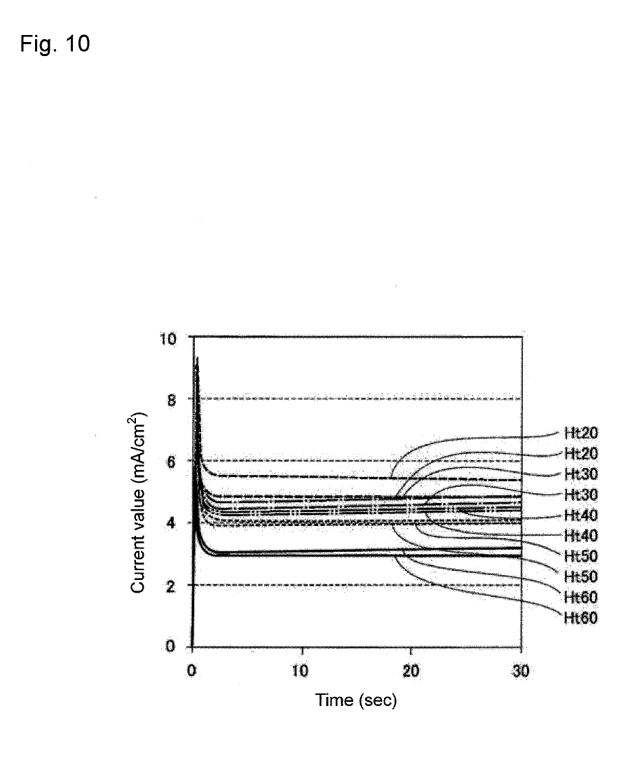

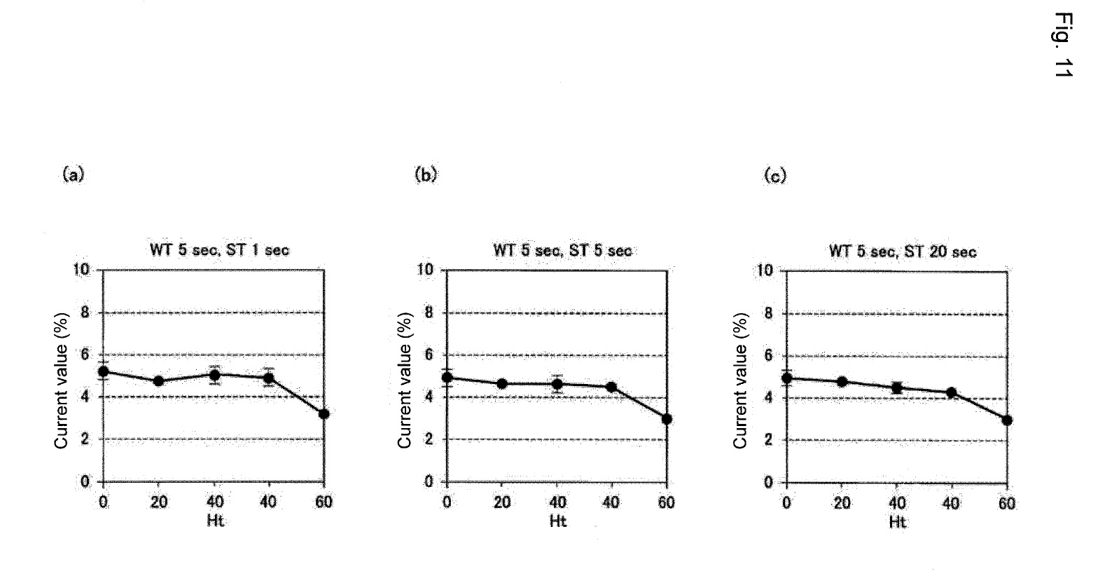

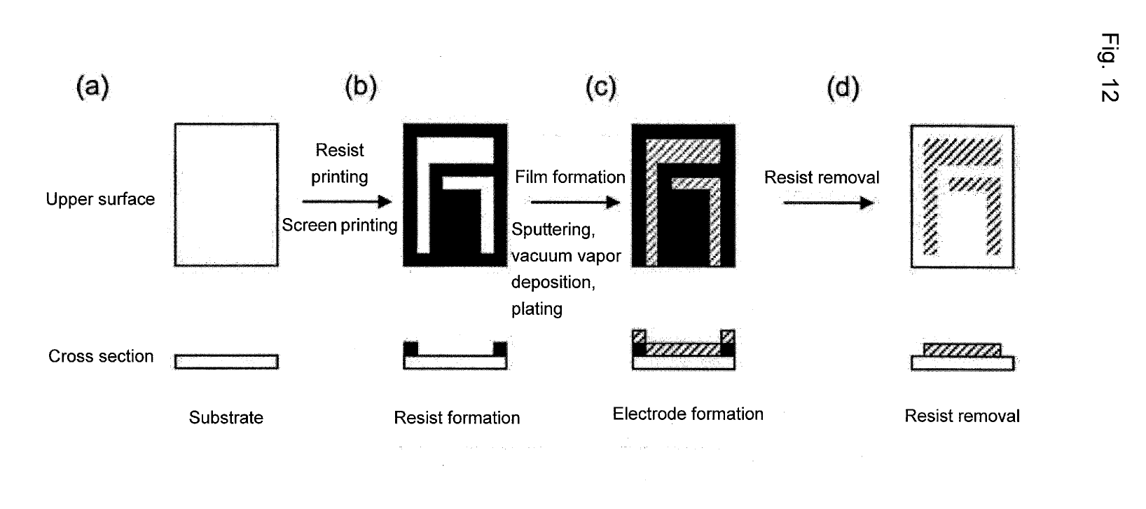

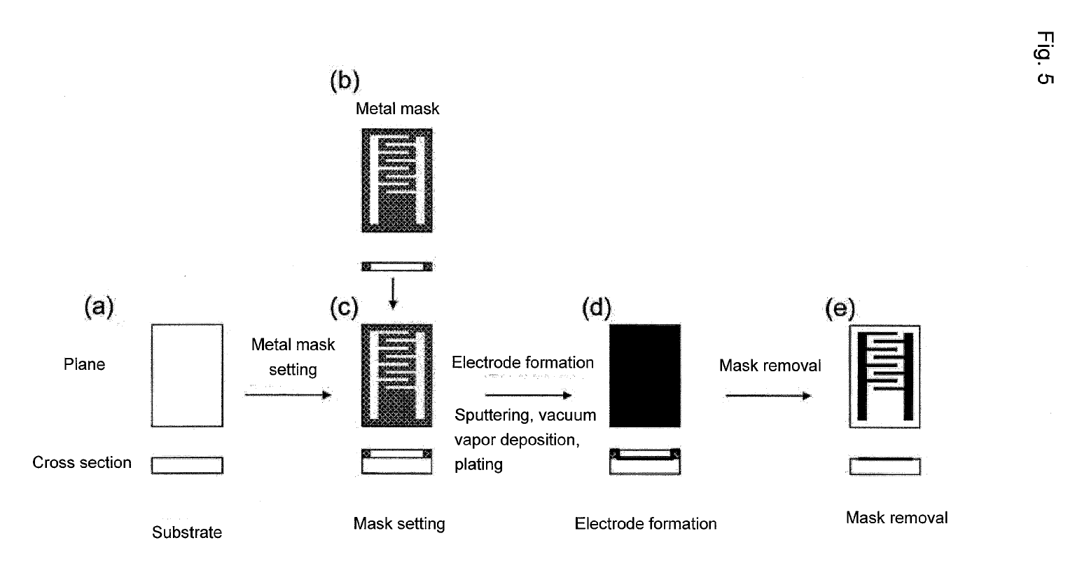

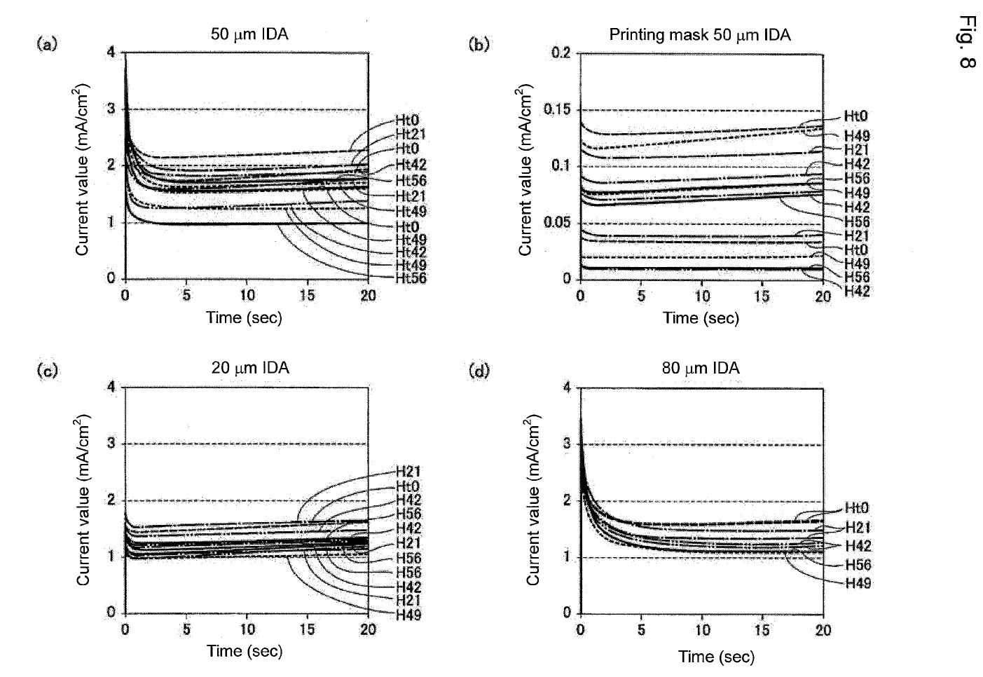

The results of chronoamperometry are shown in FIG. 10, and the effect of Ht calculated from FIG. 10 is shown in FIGS. 11(a) to 11(c). The shapes of curves of amperograms showed curves reaching a plateau region immediately after applying the potential. In the evaluation of the effect of Ht, the effect of Ht was small, and the effect was about ±10% in the range between Ht20 and Ht50.

While the present invention is herein described in detail with reference to specific embodiments, it will be apparent to those skilled in the art that various modifications and variations can be made without departing from the spirit and scope of the invention. The present Application is based on Japanese Patent Application (Japanese Patent Application No. 2013-006561) filed on Jan. 17, 2013, the entire contents of which are incorporated herein by reference.

### DESCRIPTION OF REFERENCE NUMERALS AND SIGNS

10: biosensor
102: electrically insulating substrate
104: interdigitated array electrode
108: spacer
109: cover film
1042: working electrode
1044: counter electrode
A: hole
C: cavity
G: inter-electrode distance
W: electrode width

1. A biosensor which oxidizes a blood component with an oxidoreductase, detects an oxidation current generated by the reaction product with an electrode and measures the blood component, wherein
the electrode is an interdigitated array electrode in which a working electrode and a counter electrode composed of a noble metal are alternately arranged,
the total area of the interdigitated array electrode is from 1.8 to 4 mm2, an inter-electrode distance is less than 50 μm, an electrode width of the working electrode is from 5 to 30 μm and an electrode width of the counter electrode is from 5 to 100 μm.

2. The biosensor according to claim 1, wherein the sum of the number of the working electrodes and the counter electrodes of the interdigitated array electrode is from 30 to 300.

3. The biosensor according to claim 1, wherein
the interdigitated array electrode is (1) formed by forming a noble metal film on an electrically insulating substrate, printing a resist in the form of an interdigitated array thereon by a screen printing method, performing etching, followed by removing the resist, or (2) formed by forming a noble metal film on an electrically insulating substrate, applying or adhering a resist thereon, performing light exposure through a photomask, etching the resist and the noble metal film in a portion other than a portion where the interdigitated array electrode is formed, followed by removing the resist in the portion where the interdigitated array electrode is formed, or (3) formed by superimposing a template from which a pattern of the interdigitated array electrode to be produced has been removed on an electrically insulating substrate, forming a noble metal film on the electrically insulating substrate through the template, followed by removing the template, or (4) formed by printing a resist in a portion where the interdigitated array electrode is not formed on an electrically insulating substrate by a screen printing method, forming a noble metal film on the electrically insulating substrate and the resist and removing the resist and the noble metal film formed on the resist.

4. The biosensor according to claim 1, wherein the blood component is glucose.

5. A method for producing a biosensor, comprising a step of forming an interdigitated array electrode, in which a working electrode and a counter electrode composed of a noble metal are alternately arranged, on an electrically insulating substrate, wherein
the total area of the interdigitated array electrode is from 1.8 to 4 mm2, an inter-electrode distance is less than 50 μm, an electrode width of the working electrode is from 5 to 30 μm, an electrode width of the counter electrode is from 5 to 100 μm and the number of the electrodes is from 30 to 300,
the step is (1) a step of forming an interdigitated array electrode by forming a noble metal film on an electrically insulating substrate, printing a resist in the form of an interdigitated array thereon by a screen printing method, performing etching, followed by removing the resist, or (2) a step of forming an interdigitated array electrode by forming a noble metal film on an electrically insulating substrate, applying or adhering a resist thereon, performing light exposure through a photomask, etching the resist and the noble metal film in a portion other than a portion where the interdigitated array electrode is formed, followed by removing the resist in the portion where the interdigitated array electrode is formed, or (3) a step of forming an interdigitated array electrode by superimposing a template from which a pattern of the interdigitated array electrode to be produced has been removed on an electrically insulating substrate, forming a noble metal film on the electrically insulating substrate through the template, followed by removing the template, or (4) a step of forming an interdigitated array electrode by printing a resist in a portion where the interdigitated array electrode is not formed on an electrically insulating substrate by a screen printing method forming a noble metal film on the electrically insulating substrate and the resist and removing the resist and the noble metal film formed on the resist.

6. The biosensor according to claim 2, wherein the interdigitated array electrode is (1) formed by forming a noble metal film on an electrically insulating substrate, printing a resist in the form of an interdigitated array thereon by a screen printing method, performing etching, followed by removing the resist, or (2) formed by forming a noble metal film on an electrically insulating substrate, applying or adhering a resist thereon, performing light exposure through a photomask, etching the resist and the noble metal film in a portion other than a portion where the interdigitated array electrode is formed, followed by removing the resist in the portion where the interdigitated array electrode is formed, or (3) formed by superimposing a template from which a pattern of the interdigitated array electrode to be produced has been removed on an electrically insulating substrate, forming a noble metal film on the electrically insulating substrate through the template, followed by removing the template, or (4) formed by printing a resist in a portion where the interdigitated array electrode is not formed on an electrically insulating substrate by a screen printing method, forming a noble metal film on the electrically insulating substrate and the resist and removing the resist and the noble metal film formed on the resist.

7. The biosensor according to claim 2, wherein the blood component is glucose.

8. The biosensor according to claim 3, wherein the blood component is glucose.
